(19) United States
(12) Patent Application Publication
(10) Pub. No.: US 2024/0293505 A1
(43) Pub. Date: Sep. 5, 2024

(54) REVERSAL OF CTL EXHAUSTION WITH ANNEXIN V

(71) Applicants: The Board of Trustees of the Leland Stanford Junior University, Stanford, CA (US); ERASMUS UNIVERSITY MEDICAL CENTER ROTTERDAM, Rotterdam (NL)

(72) Inventors: Francis Gerard Blankenberg, Portola Valley, CA (US); Peter D. Katsikis, Rotterdam (NL)

(21) Appl. No.: 18/282,152

(22) PCT Filed: Mar. 29, 2022
(86) PCT No.: PCT/US2022/022314

§ 371 Date: Sep. 14, 2023

Related U.S. Application Data
(60) Provisional application No. 63/167,392, filed on Mar. 29, 2021.

(51) Int. Cl. A61K 38/17

(52) U.S. Cl. CPC A61K 38/1709

(57) ABSTRACT

Methods are provided for treating an individual with cytotoxic T lymphocyte exhaustion by administering an effective dose of an annexin V agent. In some embodiments, the individual undergoing treatment is infected with HIV. The individual may be treated with highly active antiretroviral therapy (HAART).

### CROSS REFERENCE TO RELATED APPLICATION

The present application claims the benefit of and priority to U.S. Provisional Patent Application No. 63/167,392, filed Mar. 29, 2021, the entire disclosure of which is hereby incorporated by reference in its entirety.

### INCORPORATION BY REFERENCE OF SEQUENCE LISTING PROVIDED AS A TEXT FILE

A Sequence Listing is provided here within in a text file, (S18-395_STAN-1521WO_Seq_Listing_ST25.txt), created on Mar. 29, 2022, and having a size of 9,000 bytes. The contents of the text file are incorporated herein by reference in its entirety.

### BACKGROUND

Major advances in antiretroviral therapy have controlled human immunodeficiency virus (HIV) viremia in most infected individuals under treatment and have resulted in a great reduction in mortality in Western countries. However, despite these advances in our understanding of HIV pathogenesis, specifically how it causes chronic immune activation and why the immune system is unable to control the virus in most people, remains unclear. Indeed, chronic immune activation is one of the hallmarks of HIV infection, characterized by expression of activation markers on lymphocytes, polyclonal B-cell expansion, increased T-cell turnover, and elevated serum levels of proinflammatory cytokines and chemokines.

Persistent virus contributes to a sustained inflammatory environment promoting accumulation of “activated/exhausted” T cells with diminished effector function. These T cells show increased expression of immunomodulatory receptors including Programmed cell death protein (PD1), Cytotoxic T Lymphocyte Associated Protein 4 (CTLA4), Lymphocyte activation gene 3 (LAG3), T cell immunoglobulin and ITIM domain (TIGIT), T cell immunoglobulin and mucin domain containing 3 (TIM3) among others. More importantly, recent reports have demonstrated that, HIV infected T cells express checkpoint receptors, contributing to their survival and promoting maintenance of the viral reservoir.

A major problem in HIV/AIDS is the inability to clear all reservoirs of virus due to CTL exhaustion/anergy. As HAART (highly active antiretroviral therapy) treatment can only inhibit viral spread and proliferation in dividing cells it can't by itself clear potential reservoirs of virus in quiescent cells such as monocytes/macrophages. Therefore, few HIV/AIDS patients even with stable asymptomatic disease ever clear their chronic infection.

Methods of blocking intracellular pathogens through reversal of cytotoxic T lymphocyte (CTL) exhaustion are of great clinical interest, and are addressed herein.

### SUMMARY OF THE INVENTION

Methods are provided for treating an individual with cytotoxic T lymphocyte exhaustion, which exhaustion may be associated with a virus infection, including HIV infection, by administering an effective dose of an annexin V agent. AnxV proteins of interest include recombinant human annexin V protein and PS binding fragments derived therefrom, and include native proteins, and mutants (e.g. annexin V-128), and derivatives thereof sequences. Annexin V protein, including proteins lacking a terminal methionine, are of particular interest.

In some embodiments of the invention, an effective dose of human AnxV is administered, e.g. by parenteral administration, locally or systemically to an individual to reduce T cell exhaustion. In some embodiments the administration is performed by continuous iv infusion; by direct lymph node injection, by sustained release formulations, and the like. The effective dose in a human may be up to about 50 μg/kg, up to about 100 μg/kg, up to about 250 μg/kg, up to about 500 μg/kg, up to about 750 μg/kg, up to about 1 mg/kg, up to about 1.5 mg/kg, up to about 2 mg/kg, up to about 5 mg/kg, up to about 7.5 mg/kg, up to about 10 mg/kg, up to about 20 mg/kg.

In some embodiments, the human AnxV is administered in a manner that provides for prolonged blood clearance of the protein, for example where the half-life of the protein in circulation is at least about 30 minutes, at least about 1 hour, at least about 1.5 hours, at least about 2 hours, at least about 2.5 hours, at least about 3 hours or more. In other embodiments the route of administration is intra-venous injection over an extended period of time, for example where a daily dosage as described above is delivered over a period of up to 30 minutes, up to one hour, up to 2 hours, up to 4 hours, up to 6 hours, up to 8 hours, up to 12 hours, up to 16 hours, up to 24 hours.

In some embodiments, the individual undergoing treatment for T cell exhaustion is infected with HIV. In some embodiments the individual is also being treated with highly active antiretroviral therapy (HAART). In some embodiments, the individual undergoing HAART treatment has an HIV viral load of less than 10,000 viral copies per milliliter of blood. For instance, the individual may have from 10,000 to 9,000 viral copies, 9,000 to 8,000 viral copies, 8,000 to 7,000 viral copies, 7,000 to 6,000 viral copies, 6,000 to 5,000 viral copies, 5,000 to 4,000 viral copies, 4,000 to 3,000 viral copies, 3,000 to 2,000 viral copies, 2,000 to 1,000 viral copies, 1,000 to 500 viral copies, 500 to 100 viral copies, 100 to 20 viral copies or less than 20 viral copies per milliliter of blood.

The effective dose of annexin V may be combined with other treatment modalities. In some embodiments a synergistic effect is observed when the annexin V therapy is combined with anti-retroviral therapy, for example highly active antiretroviral therapy.

Another aspect of the present invention relates to the use of an annexin V agent in the manufacture of a medicament to treat infection with HIV.

In some embodiments the number of exhausted (anergic) T cells in the individual are reduced following treatment. In some embodiments the individual is assessed for the decrease in specific T cell exhaustion, e.g. by determining the proliferation of HIV-specific CTL expansion in response to antigen; by measuring an increase in granzyme B in HIV-specific CTL, etc. In some embodiments the effect is determined to be specific for HIV T cell exhaustion.

In some embodiments, where the individual being treated is infected with HIV, the annexin treatment reduces the HIV viral load, e.g. by at least 10%, at least 20%, at least 30%, at least 40%, at least 50%, at least 60%, at least 70%, at least 80%, at least 90%, at least 95%, at least 99% or more. In some embodiments the number of infected T cells is reduced.

Still another aspect of the present invention provides a kit to treat infection with HIV. The kit includes a therapeutic annexin V agent, which blocks PS on the surface of infected cells in an amount sufficient to reduce T cell exhaustion. The kit may also include anti-retroviral agents. The kit may also instructions for use, reagents for monitoring intracellular pathogen growth, and the like.

### BRIEF DESCRIPTION OF THE DRAWINGS

The invention is best understood from the following detailed description when read in conjunction with the accompanying drawings. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. HIV Gag-specific CD8+ T cells after stimulation for 5 days in culture with HIV Gag peptide with or without treatment with Annexin V or blocking antibodies to CTLA4 or PD-L1. Representative FACS plots shown. FACS assay after incubation of peripheral blood lymphocytes from a patient with HIV with fluorescent GAG/HLA-1 tetramer and anti-CD8 antibody. GAG/HLA-1 tetramer binds to CTLs which recognize the HIV Gag viral protein co-presented with HLA-1 antigen (using the viral antigen-HLA-1 tetramer complex to simulate an infected cell). Anti-CD8 antibody marks CD8+ lymphocytes which are the most common type of cytotoxic T-cell. High double fluorescent staining indicates HIV-specific/CD8+ lymphocytes (marked by boxes, with the percentages of total lymphocyte population in the sample). HIV peptide=(Peptide), IgG antibody=(isotype) antibody as a control for anti-PD-L1 and anti-CTLA-4 antibody treatment. AnnV=annexin V.

FIG. 2A-2D. Percentage and absolute numbers of HIV Gag-specific CD8+ T cells after culture for 5 days with HIV Gag peptide with or without treatment with Annexin V or blocking antibodies to CTLA4 or PD-L1. A) The percentages, B) fold increase of percentage, C) absolute numbers and D) fold increase of absolute number of HIV-specific CTLs of nine different HIV patients in response to HIV peptide with and without annexin V, anti-PD-1 & anti-CTLA-4 antibody immune checkpoint inhibition.

FIG. 3. Dilution of proliferation dye with decreased fluorescence (dilution of probe, leftward shift of curve) as the HIV-specific CTL population expands in response to HIV-peptide.

FIG. 4. The CD25 antigen represents the α-chain of the IL-2 receptor which is expressed on activated T cells and therefore is a direct marker of T cell activation in this case of HIV-specific CTLs. Data from nine different HIV patients shown.

FIG. 5. The activation of HIV-specific CTLs by annexin V results in significant increases in both the number of Granzyme B positive CTLs as well as an increase its total amount. Granzyme B is a serine protease most commonly found in the granules of cytotoxic lymphocytes (CTLs), natural killer cells (NK cells) and cytotoxic T cells. It is secreted by these cells along with the pore forming protein perforin to mediate apoptosis in target cells.

FIG. 6. CMV-specific CTLs in HIV patients are not exhausted (i.e. anergic) and untreated CMV-specific CTLs proliferate to the same degree as treated CMV-specific CTLs in response to CMV antigen.

### DETAILED DESCRIPTION OF THE INVENTION

The disclosure provides methods to reverse exhaustion of cytotoxic T cells by administering to the subject an effective amount of an agent that provides annexin V binding activity. In some embodiments the T cell exhaustion is related to HIV infection, including without limitation treatment of the HIV infection with HAART. Administration of the annexin V compositions can be performed using any of the various methods and delivery systems known to those skilled in the art. The delivery systems described below, which employ a number of routinely used pharmaceutical carriers, are only representative of the many embodiments envisioned for administering the annexin V compositions.

Annexin-V (PAP-I, lipocortin-V) acts as a potent anticoagulant by binding to negatively charged phospholipids with high affinity, for example having a Kd in the 10⁻⁹ to 10⁻¹⁰ M range. Annexin V forms a shield around negatively-charged phospholipid molecules. The formation of blocks the entry of phospholipids into coagulation (clotting) reactions, and prevents interaction of the phospholipid with immunoregulatory cells. The genetic sequence of human annexin V can be accessed at Genbank, NM_001154. The crystal and molecular structure is described in Romisch and Paques (1992) J. Mol. Biol. 223 (3), 683-704. Annexin V polypeptides or biologically active fragments and variants thereof, and the like, are used in the treatment of cancer. In some embodiments the annexin V has a wild-type or native sequence.

The Annexin A5 or the functional analog or variant thereof according to the invention can be human Annexin A5 (SEQ ID NO:2), an allelic or genetic variant thereof, a mammalian orthologue thereof, or an allelic or genetic variant thereof. The coding sequence is provided as SEQ ID NO:1. Preferably the functional analog or variant of Annexin A5 according to the invention is more than 50%, 60%, 70%, 75%, such as more than 80% or 85%, more than 90%, or preferably more than 95% or 99% identical to human Annexin A5, SEQ ID NO:2. The Annexin V can lack a terminal methionine.

The protein sequence of human annexin V is MAQVLRGTVTDFPGFDERADAETLRKAMKGLGTDEESILTLLTSRSNAQRQEISAAFKTLFGR DLLDDLKSELTGKFEKLIVALMKPSRLYDAYELKHALKGAGTNEKVLTEIIASRTPEELRAIKQVY EEEYGSSLEDDVVGDTSGYYQRMLVVLLQANRDPDAGIDEAQVEQDAQALFQAGELKWGTD EEKFITIFGTRSVSHLRKVFDKYMTISGFQIEETIDRETSGNLEQLLLAVVKSIRSIPAYLAETLYY AMKGAGTDDHTLIRVMVSRSEIDLFNIRKEFRKNFATSLYSMIKGDTSGDYKKALLLLCGEDD (SEQ ID NO:2).

In other embodiments the annexin V is an annexin V-128 mutant protein. When the annexin V is an annexin V-128 mutant protein, it has the following protein sequence: MAQVLRGTVTDFPGFDERADAETLRKAMKGLGTDEESILTLLTSRSNAQRQEISAAFKTLFGR DLLDDLKSELTGKFEKLIVALMKPSRLYDAYELKHALKGAGTNEKVLTEIIASRTPEELRAIKQVY EEEYGSSLEDDVVGDTSGYYQRMLVVLLQANRDPDAGIDEAQVEQDAQALFQAGELKWGTD EEKFITIFGTRSVSHLRKVFDKYMTISGFQIEETIDRETSGNLEQLLLAVVKSIRSIPAYLAETLYY AMKGAGTDDHTLIRVMVSRSEIDLFNIRKEFRKNFATSLYSMIKGDTSGDYKKALLLLCGEDDA GGCGH (SEQ ID NO: 3).

A functional analog or variant of Annexin A5 may be a protein wherein at one or more positions there have been amino acid insertions, deletions, or substitutions, either conservative or non-conservative, provided that such changes result in a protein whose basic properties to function in an equivalent manner to Annexin A5 have not significantly been changed. “Significantly” in this context means that one skilled in the art would say that the properties of the variant may still be different but would not be unobvious over the ones of the original protein. By “conservative substitutions” is intended combinations such as Gly, Ala; Val, Ile, Leu; Asp, Glu; Asn, Gln; Ser, Thr; Lys, Arg; and Phe, Tyr. Such variants may be made using the methods of protein engineering and site-directed mutagenesis which are well known in the art.

The functional analog or variant of Annexin A5 according to the invention may, or may not, be a dimer of Annexin A5 or a functional analog or variant thereof, or may or may not, be a PEGylated Annexin A5 or a functional analog or variant thereof. DiAnnexinA5 and PEGylated AnnexinA5 are disclosed in WO 02/067857.

PEGylation is a method well known to those skilled in the art wherein a polypeptide or peptidomimetic compound (for the purposes of the present invention, Annexin V or the functional analog or variant) is modified such that one or more polyethylene glycol (PEG) molecules are covalently attached to the side chain of one or more amino acids or derivatives thereof. It is one of the most important molecule altering structural chemistry techniques (MASC). Other MASC techniques may be used; such techniques may improve the pharmacodynamic properties of the molecule, for example extending its half life in vivo. A PEG-protein conjugate is formed by first activating the PEG moiety so that it will react with, and couple to, the protein or peptidomimetic compound of the invention. PEG moieties vary considerably in molecular weight and conformation, with the early moieties (monofunctional PEGs; mPEGs) being linear with molecular weights of 12 kDa or less, and later moieties being of increased molecular weights. PEG2, a recent innovation in PEG technology, involves the coupling of a 30 kDa (or less) mPEG to a lysine amino acid (although PEGylation can be extended to the addition of PEG to other amino acids) that is further reacted to form a branched structure that behaves like a linear mPEG of much greater molecular weight (Kozlowski et al., (2001), Biodrugs 15, 419-429). Methods that may be used to covalently attach the PEG molecules to polypeptides are further described in Roberts et al., (2002) Adv Drug Deliv Rev, 54, 459-476, Bhadra et al., (2002) Pharmazie 57, 5-29, Kozlowski et al., (2001) J Control Release 72, 217-224, and Veronese (2001) Biomaterials 22, 405-417 and references referred to therein.

The advantages of PEGylation to the polypeptide or peptidomimetic compound of the invention include reduced renal clearance which, for some products, results in a more sustained adsorption after administration as well as restricted distribution, possibly leading to a more constant and sustained plasma concentrations and hence an increase in clinical effectiveness (Harris et al., (2001) Clin Pharmacokinet 40, 539-551). Further advantages can include reduced immunogenicity of the therapeutic compound (Reddy, (2001) Ann Pharmacother 34, 915-923), and lower toxicity (Kozlowski et al., (2001), Biodrugs 15, 419-429).

The functional analog or variant of Annexin A5 according to the invention can be a fusion protein comprising the sequence of Annexin A5 or a variant thereof. Thus, for example, Annexin A5 or a variant thereof can be fused to one or more fusion partner polypeptide sequence(s) so as to extend the half-life of the molecule within a patient's circulatory system and/or add further functionality to the molecule.

By a “functional” analog or variant of Annexin A5 is meant a protein capable of binding to phosphatidylserine on a biological membrane, preferably to a level that is at least 10%, 20%, 30%, 40%, 50%, 60%, 70%, 80%, 90%, 95%, 99% or about 100% of that displayed by human Annexin A5 (SEQ ID NO:2) under the same conditions. Suitable method for measuring Annexin A5 binding to phosphatidylserine on a biological membrane are known in the art.

A “functional” analog or variant of Annexin A5 may, additionally, or alternatively, also possess at least 10%, 20%, 30%, 40%, 50%, 60%, 70%, 80%, 90%, 95%, 99% or about 100% of the therapeutic activity human Annexin V (SEQ ID NO:2) when used at the same (i.e. molar equivalent) dosage, for blocking or reversing EV mediated transcriptional changes.

Annexin V polypeptides, which can be used in the methods of the invention, comprise at least about 50 contiguous amino acids, usually at least about 100 contiguous amino acids, at least about 150 contiguous amino acids, at least about 200 contiguous amino acids, at least about 250 contiguous amino acids, and which may include up to the full length of native annexin V protein, including without limitation human annexin V protein, or modifications thereof, and may further include fusion polypeptides as known in the art in addition to the provided sequences.

In one embodiment of the invention, the Annexin V polypeptide consists essentially of a polypeptide sequence of around about 320 amino acids in length and having a sequence of a native Annexin V protein, or an Annexin V protein lacking a terminal methionine as described above. By “consisting essentially of” in the context of a polypeptide described herein, it is meant that the polypeptide is composed of the Annexin V sequence, which sequence is optionally flanked by one or more amino acid or other residues that do not materially affect the basic characteristic(s) of the polypeptide.

A pharmaceutical composition comprises Annexin V or a functional analog or variant thereof in admixture with a pharmaceutically or veterinarily acceptable adjuvant, diluent or carrier, which will typically be selected with regard to the intended route of administration and standard pharmaceutical practice. The composition may be in the form of immediate-, delayed- or controlled-release applications. Preferably, the formulation is a unit dosage containing a daily dose or unit, daily sub-dose or an appropriate fraction thereof, of the active ingredient.

The pharmaceutical composition according to the invention may, or may not, be intended for, and, thus formulated in a manner suitable for, parenteral, intravenous, intra-arterial, intraperitoneal, intra-muscular or subcutaneous administration, or they may be administered by infusion techniques. They may be best used in the form of a sterile aqueous solution which may contain other substances, for example, enough salts or glucose to make the solution isotonic with blood. The aqueous solutions may be suitably buffered (preferably to a pH of from 3 to 9), if necessary. The preparation of suitable pharmaceutical formulations under sterile conditions is readily accomplished by standard pharmaceutical techniques well-known to those skilled in the art.

Such formulations may include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

A therapeutically effective amount of Annexin V or a functional analog or variant thereof for administration to a patient, such as a human patient, on the basis of a daily dosage level may be from 0.01 to 1000 mg of Annexin V or a functional analog or variant thereof per adult (for example, from about 0.001 to 20 mg per kg of the patient's body weight, such as 0.01 to 10 mg/kg, for example greater than 0.1 mg/kg and less than 20, 10, 5, 4, 3 or 2 mg/kg, such as about 1 mg/kg), administered in single or divided doses.

The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The annexin V for use in the subject methods may be produced from eukaryotic or prokaryotic cells, or may be synthesized in vitro. Where the protein is produced by prokaryotic cells, it may be further processed by unfolding, e.g. heat denaturation, DTT reduction, etc. and may be further refolded, using methods known in the art. By using synthesizers, naturally occurring amino acids may be substituted with unnatural amino acids. The particular sequence and the manner of preparation will be determined by convenience, economics, purity required, and the like.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The terms “treatment”, “treating”, “treat” and the like are used herein to generally refer to obtaining a desired pharmacologic and/or physiologic effect. The effect can be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. Those in need of treatment include those already with an infection as well as those in which an infection is to be prevented. As such, a therapeutic treatment is one in which the subject is infected prior to administration and a prophylactic treatment is one in which the subject is not infected prior to administration. In some embodiments, the subject is suspected of being infected prior to administration. In some embodiments, the subject has an increased risk of infection prior to administration. In some embodiments, the subject is suspected of being at increased risk of infection prior to administration.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of Annexin V is an amount that is sufficient to palliate, ameliorate, stabilize, reverse, prevent, slow or delay the progression of the disease state (e.g., virus) by decreasing T cell exhaustion.

As used herein, a “target cell” may be an HIV infected cell. Usually a target cell is a mammalian cell, for example a human cell.

As used herein, the term “infection” refers to any state in at least one cell of an organism (i.e., a subject) is infected by an infectious agent. As used herein, the term “infectious agent” refers to a foreign biological entity, i.e. a pathogen, that utilizes phosphatidylserine to decrease host immune responses. For the present disclosure the pathogen may be human immunodeficiency virus (HIV).

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to contact an entity on the cell surface and bind to it. In one embodiment, the term “suitable conditions” as used herein means physiological conditions.

The terms “specific binding,” “specifically binds,” and the like, refer to non-covalent or covalent preferential binding to a molecule relative to other molecules or moieties in a solution or reaction mixture (e.g., annexin V specifically binds to phosphatidylserine). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}. In an embodiment, affinity is determined by surface plasmon resonance (SPR), e.g. as used by Biacore systems. The affinity of one molecule for another molecule is determined by measuring the binding kinetics of the interaction, e.g. at 25° C.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sample” with respect to a patient encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as intracellular pathogen cells. The definition also includes samples that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample comprising target cells or normal control cells or suspected of comprising such cells or biological fluids derived therefrom (e.g., a pathogen, an infected cell, etc.), e.g., a sample comprising polynucleotides and/or polypeptides that is obtained from such cells (e.g., a cell lysate or other cell extract comprising polynucleotides and/or polypeptides). A biological sample comprising an inflicted cell from a patient can also include non-inflicted cells.

Phosphatidylserine (PS) usually resides in the inner leaf of the cell membrane. When a cell dies (either by apoptosis, necroptosis, or pyroptosis), PS is exposed on the surface of the cell membrane. The exposed PS is recognized by PS-binding proteins of either soluble proteins or cell surface receptors, which can mediate phagocytic removal of PS-exposing cells by phagocytes such as macrophages. Viral infection, including Influenza virus and HIV-1, can induce cell death and exposure of PS. PS-dependent phagocytic removal of Influenza virus-infected cells has been shown to inhibit viral replication in in vitro and in vivo settings. Such apoptosis-dependent phagocytic removal of infected cells has been seen with HIV-1 infection.

At the virus binding and entry steps, interaction of the envelope PS and the host's PS-binding molecules can enhance HIV-1 infection of cells by facilitating virus attachment. At the virus budding step, HIV-1 can be trapped on the cell surface by one family of PS-binding receptors, T-cell immunoglobulin mucin domain proteins (TIM)-1, 3, and 4 expressed on virus producer cells. Although this trapping can inhibit release of HIV-1, one of the HIV-1 accessory gene products, Negative Factor (Nef), can counteract virus trapping by TIM family receptors (TIMs) by inducing the internalization of these receptors. HIV-1 infection can induce exposure of PS on infected cells by inducing cell death. A soluble PS-binding protein in serum, protein S, bridges PS exposed on HIV-1-infected cells and a receptor tyrosine kinase, Mer, expressed on macrophages and mediate phagocytic clearance of HIV-1 infected cells. HIV-1 can also induce exposure of PS on target cells at the virus binding step. Binding of HIV-1 envelope proteins to its receptor (CD4) and co-receptors (CXCR4 or CCR5) elicit signals that induce PS exposure on target cells by activating TMEM16F, a phospholipid scramblase. PS exposed on target cells enhances HIV-1 infection by facilitating fusion between the viral envelope and target cell membrane.

The human immunodeficiency viruses (HIV) are two species of Lentivirus (a subgroup of retrovirus) that infect humans. Over time, they cause acquired immunodeficiency syndrome (AIDS), a condition in which progressive failure of the immune system allows life-threatening opportunistic infections and cancers to thrive.

HIV infects vital cells in the human immune system, such as helper T cells (specifically CD4+ T cells), macrophages, and dendritic cells. HIV infection leads to low levels of CD4+ T cells through a number of mechanisms, including pyroptosis of abortively infected T cells, apoptosis of uninfected bystander cells, direct viral killing of infected cells, and killing of infected CD4+ T cells by CD8+ cytotoxic lymphocytes that recognize infected cells. When CD4+ T cell numbers decline below a critical level, cell-mediated immunity is lost, and the body becomes progressively more susceptible to opportunistic infections, leading to the development of AIDS.

Two types of HIV have been characterized: HIV-1 and HIV-2. HIV-1 is the virus that was initially discovered and termed both lymphadenopathy associated virus (LAV) and human T-lymphotropic virus 3 (HTLV-III). HIV-1 is more virulent and more infective than HIV-2, and is the cause of the majority of HIV infections globally. The lower infectivity of HIV-2, compared to HIV-1, implies that fewer of those exposed to HIV-2 will be infected per exposure. Due to its relatively poor capacity for transmission, HIV-2 is largely confined to West Africa.

HIV is roughly spherical with a diameter of about 120 nm, around 60 times smaller than a red blood cell. It is composed of two copies of positive-sense single-stranded RNA that codes for the virus's nine genes enclosed by a conical capsid composed of 2,000 copies of the viral protein p24. The single-stranded RNA is tightly bound to nucleocapsid proteins, p7, and enzymes needed for the development of the virion such as reverse transcriptase, proteases, ribonuclease and integrase. A matrix composed of the viral protein p17 surrounds the capsid ensuring the integrity of the virion particle.

The RNA genome consists of at least seven structural landmarks (LTR, TAR, RRE, PE, SLIP, CRS, and INS), and nine genes (gag, pol, and env, tat, rev, nef, vif, vpr, vpu, and sometimes a tenth tev, which is a fusion of tat, env and rev), encoding 19 proteins. Three of these genes, gag, pol, and env, contain information needed to make the structural proteins for new virus particles. For example, env codes for a protein called gp160 that is cut in two by a cellular protease to form gp120 and gp41. The six remaining genes, tat, rev, nef, vif, vpr, and vpu (or vpx in the case of HIV-2), are regulatory genes for proteins that control the ability of HIV to infect cells, produce new copies of virus (replicate), or cause disease.

Cytotoxic T-lymphocyte (CTL) responses have been indicated to play an important role in the control of HIV and simian immunodeficiency virus (SIV) infections. Above all, the potential of Gag-specific CTL responses to contribute to viral control has been suggested by a cohort study indicating an association of HIV control with the breadth of Gag-specific CTL responses.

Granzyme B is a 32-kDa serine protease resembling chymotrypsin, and has homologues expressed in a number of different species. The gene product encoding GrB is ˜3500 bp long, contains five exons and four introns, and maps to chromosome 14 on the human genome. Similar to caspases, GrB has a preference for cleaving peptides immediately adjacent to aspartate (Asp) residues. This specificity is due to the structure of the GrB active site, which contains an arginine (Arg) residue positioned at the side of the active site pocket. An interaction between an Asp residue at the P1 position of the substrate and the Arg residue within the active site is key for enzyme-substrate interaction. Transcriptional activation of GrB within T lymphocytes involves activation of the T cell receptor and co-stimulation with cytokines. Most lymphocytes constitutively express GrB transcripts and upregulate transcription when the lymphocyte has been activated. GrB is known to be an inducer of cell death. This function is established within the cytoplasm of target cells GrB internalization is facilitated by perforin. Upon internalization, GrB initiates apoptosis primarily through the cleavage of Bid into a truncated form (gtBid) that triggers mitochondrial cytochrome c release and apoptosome formation leading to caspase activation and manifestation of the apoptosis phenotype. GrB can also bypass the mitochondrial pathway and initiate caspase activation directly and/or cleave caspase substrates such as the inhibitor of caspase-activated deoxyribonuclease (ICAD), thereby allowing CAD to translocate to the nucleus to fragment DNA. GrB also cleaves the nuclear membrane protein lamin B, resulting in a loss of integrity of the nuclear membrane.

As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, protein therapeutics are commonly administered parenterally (e.g., by intravenous or subcutaneous injection).

As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in an underlying DNA sequence without affecting the protein sequence. Moreover, due to biological functional equivalency considerations, changes, particularly conservative changes, can be made in protein structure without affecting the biological action in kind or amount. All such modifications are intended to be included within the scope of the appended claims.

### Methods

Methods are provided for treating or reducing infection, including without limitation, viral infections, associated with cytotoxic T lymphocyte (CTL) exhaustion by blocking PS on surface of infected cells, thereby reversing CTL exhaustion and preventing suppression of host immune responses. Such methods include administering to a subject in need of treatment a therapeutically effective amount or an effective dose of annexin V, including without limitation combinations of the reagent with another drug. In one aspect, the present invention discloses a method for treating viral infection by reducing immunosuppression associated with the infection.

T-cell exhaustion is characterized by the stepwise and progressive loss of T-cell functions and can culminate in the physical deletion of the responding cells. Exhaustion is well-defined during chronic lymphocytic choriomeningitis virus infection and commonly develops under conditions of antigen-persistence, which occur following many chronic infections that are of significant public health concern including hepatitis B virus, hepatitis C virus and human immunodeficiency virus infections, as well as during tumor outgrowth. Exhaustion is not a uniformly disabled setting as a gradation of phenotypic and functional defects can manifest, and these cells are distinct from prototypic effector, memory and also anergic T cells.

The use of multiple drugs that act on different viral, e.g. HIV, targets is known as highly active antiretroviral therapy (HAART). HAART decreases the patient's total burden of HIV, maintains function of the immune system, and prevents opportunistic infections that often lead to death. HAART also prevents the transmission of HIV between serodiscordant same sex and opposite sex partners so long as the HIV-positive partner maintains an undetectable viral load.

Currently, the standard of care for a treatment-naïve patient with HIV-1 is a three-drug, HAART regimen that is started as soon as possible after a patient tests positive for HIV. A foundation of HAART is the administration of drugs that inhibit HIV viral replication at several stages in the lifecycle through different mechanisms to prevent viral resistance to any single agent. However, the selection of these drugs and the life-long treatment of a patient with HIV can be complex. Management of a HAART regimen is a multifaceted process that should be administered by, or in consultation with, a provider with specific training as defined by the HIV-Medicine Association of the Infectious Diseases Society of America. This approach is crucial to optimize patient care as studies have demonstrated provider experience positively correlates with improved patient outcomes. Healthcare professionals should work as a team to ensure they have a comprehensive patient history before selecting a HAART combination. Additionally, a board-certified infectious disease pharmacist who specializes in HIV and HAART is an invaluable consulting member of the interprofessional healthcare team. Nurses will help monitor therapy, note progress or lack thereof, and verify patient compliance with therapy, which is of paramount importance with HAART. As always, healthcare team coordination and patient education are critical components to maximize patient adherence, prevent further spread of disease, and provide continuity of care to the patients. Interprofessional teamwork will optimize the therapeutic benefit of HAART. Current standards of care of HIV HAART based treatments has been described in Eggleton et al. (2020) StatPearls [Internet] which has been incorporated herein by reference.

There are six main classes of HAART agents that target different stages in the viral lifecycle. A fundamental cornerstone of HAART is the co-administration of different drugs that inhibit HIV replication by several mechanisms so that the propagation of a virus with resistance to a single agent is inhibited by the action of the other two agents. Some agents may be co-formulated to increase ease of patient compliance with these medications. The six main classes are as follows: Nucleoside/Nucleotide Reverse Transcriptase Inhibitors (NRTIs), Non-nucleoside Reverse Transcriptase Inhibitors (NNRTIs), Protease inhibitors (PIs), Integrase Strand Transfer Inhibitors (INSTIs), Fusion inhibitors (FIs) and Chemokine Receptor Antagonists (CCR5 Antagonists)

NRTIs require intracellular phosphorylation via host enzymes before they can inhibit viral replication. These agents are nucleoside or nucleotide analogs with an absent hydroxyl at the 3′ end that are incorporated into the growing viral DNA strand. They competitively bind to reverse transcriptase and cause premature DNA chain termination as they inhibit 3′ to 5′ phosphodiester bond formation. Suitable NRTIs that find use in the present disclosure include, without limitation, abacavir, didanosine, lamivudine, stavudine, tenofovir, zidovudine, etc.

NNRTIs bind to HIV reverse transcriptase at an allosteric, hydrophobic site. These agents cause a stereochemical change within reverse transcriptase, thus inhibiting nucleoside binding and inhibition of DNA polymerase. Suitable NNRTIs that find use in the present disclosure include, without limitation, delavirdine, efavirenz, nevirapine, rilpivirine, etc.

PIs competitively inhibit the proteolytic cleavage of the gag/pol polyproteins in HIV-infected cells. These agents result in immature, non-infectious virions. PIs are generally used in patients who fail their initial HAART regimen and should be administered with boosting agents such as ritonavir or cobicistat. Suitable PIs that find use in the present disclosure include, without limitation, atazanavir, darunavir, indinavir, etc.

INSTIs bind viral integrase and prevent viral DNA from being incorporated into the host cell chromosome. Suitable INSTIs that find use in the present disclosure include, without limitation, dolutegravir, elvitegravir, raltegravir, etc.

FIs bind to the envelope glycoprotein gp41 and prevent viral fusion to the CD4 T-cells. Suitable FIs that find use in the present disclosure include, without limitation, maraviroc, aplaviroc, vicriviroc, etc.

CCR5 antagonists selectively and reversibly block entry into the CD4 T-cells by preventing interaction between CD4 cells and the gp120 subunit of the viral envelope glycoprotein. Suitable CCR5 antagonists that find use in the present disclosure include, without limitation, maraviroc, enfuvirtide, etc.

In some embodiments the infection is a chronic HIV infection, i.e. an infection that is not cleared by the host immune system within a period of up to 1 week, 2 weeks, etc.

The effective daily dose of an annexin V agent, e.g. human annexin V protein, in a human may be up to about 50 μg/kg, up to about 100 μg/kg, up to about 250 μg/kg, up to about 500 μg/kg, up to about 750 μg/kg, up to about 1 mg/kg, up to about 1.5 mg/kg, up to about 2 mg/kg, up to about 5 mg/kg, up to about 7.5 mg/kg, up to about 10 mg/kg, up to about 20 mg/kg. The administration of a therapeutically effective dose of an annexin V agent can be achieved in a number of different ways. Suitable administration of a therapeutically effective dose can entail administration of a single dose, or can entail administration of doses daily, semi-weekly, weekly, once every two weeks, once a month, annually, etc., including continuous infusion.

In some embodiments, the methods of the invention involve diagnosis of a patient as suffering from HIV infection; treating the patient with a regimen of annexin V therapy, optionally in combination with an additional therapy; and monitoring the patient for efficacy of treatment. Monitoring may measure clinical indicia of infection, e.g. fever, white blood cell count, etc., and/or direct monitoring for presence of the pathogen; determining efficacy on reversing CTL exhaustion, and the like. A pathogen infection can be monitored during and after treatment by the methods of the present invention. Clinical efficacy can be measured by any method known in the art. In some embodiments, clinical efficacy of the subject treatment method is determined by measuring the clinical benefit rate (CBR).

In some embodiments, the effective dose of annexin V results in an increase in the expression of Granzyme B in CTLs. For instance, an effective dose of annexin V may increase Granzyme B expression by about 10%, about 15%, about 20/%, about 25%, about 30%, about 35%, about 40%, about 45%, about 50%, about 55%, about 60%, about 65%, about 70%, about 75%, about 80%, about 85%, about 90%, about 95%, about 100% or greater than about 100% relative to the absence of annexin V treatment. The increase in Granzyme B expression is optionally determined in HIV-specific T cells.

In some embodiments, the effective dose of annexin V results in an increase in the expression of CD25 in CTLs. For instance, an effective dose of annexin V may increase CD25 expression by about 10%, about 15%, about 20/%, about 25%, about 30%, about 35%, about 40%, about 45%, about 50%, about 55%, about 60%, about 65%, about 70%, about 75%, about 80%, about 85%, about 90%, about 95%, about 100% or greater than about 100% relative to the absence of annexin V treatment. The increase in CD25 expression is optionally determined in HIV-specific T cells.

In some embodiments, the effective dose of annexin V results in an increase in the expansion of CTLs. For instance, an effective dose of annexin V may increase CTL expansion by about 1%, about 2%, about 3%, about 4%, about 5%, about 6%, about 7%, about 8%, about 9%, about 10%, about 15%, about 20/%, about 25%, about 30%, about 35%, about 40%, about 45%, about 50%, about 55%, about 60%, about 65%, about 70%, about 75%, about 80%, about 85%, about 90%, about 95%, about 100% or greater than about 100% relative to the absence of annexin V treatment. In some embodiments, the CTL expansion is HIV specific.

### Pharmaceutical Compositions

Suitable annexin V agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an annexin V agent includes use in combination with another therapeutic agent (e.g., another anti-pathogen agent). Therapeutic formulations comprising one or more annexin V agents of the invention are prepared for storage by mixing the agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The annexin V agent can be administered by any suitable means, particularly parenteral. Parenteral infusions include intramuscular, intravenous (bolus or slow drip), intraarterial, intraperitoneal, intrathecal, intra-intracellular pathogen, or subcutaneous administration.

The annexin V agent need not be, but is optionally formulated with one or more agents that potentiate activity, or that otherwise increase the therapeutic effect. These are generally used in the same dosages and with administration routes as used herein before or about from 1 to 99% of the heretofore employed dosages.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear annexin V by non-covalent associations, such as non-covalent bonding or by encapsulation. The nature of the carrier can be either soluble or insoluble for purposes of the invention. Those skilled in the art will know of other suitable carriers or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the annexin V agent through an acceptable chemical linker or chelation carrier. Positron emitting moieties for use in the present invention include ¹⁸F, which can be easily conjugated by a fluorination reaction with the annexin V agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the annexin V agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing a therapeutic dosage range and/or a priming dosage range for use in humans. The exact formulation, route of administration and dosage can be chosen by the individual physician in view of the patient's condition.

### Kits

Also provided are kits for use in the methods. The subject kits include an annexin V agent, e.g. full-length human annexin V protein or active fragment derived therefrom. In some embodiments, the agent is provided in a dosage form (e.g., a therapeutically effective dosage form). In some embodiments, annexin V is provided in two or more different dosage forms (e.g., two or more different therapeutically effective dosage forms). In the context of a kit, an agent can be provided in liquid or sold form in any convenient packaging (e.g., stick pack, dose pack, etc.).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site. The kit may further comprise imaging agents for detection and imaging of PS positive intracellular pathogen cells suitable for treatment with the methods of the invention.

The invention now being fully described, it will be apparent to one of ordinary skill in the art that various changes and modifications can be made without departing from the spirit or scope of the invention.

### EXPERIMENTAL

Our invention is the use of exogenously administered annexin V, a human 36 kDa protein with a nanomolar affinity for membrane bound PS, to mask the highly immunosuppressive effects of externalized PS and to decrease PS-mediated enhancement of diacylglycerol kinase (DGK) activity within HIV specific T cells.

Annexin V is a ubiquitous 36 kDa intracellular protein which binds to externalized PS with high nanomolar affinity in the presence of extracellular concentrations of ionized calcium. Annexin V is the smallest membrane of the annexin family of proteins and consists only of four conserved calcium dependent PS binding cores without a significant N-terminus. The longer N-terminal domains found in the other members of the annexin family typically bind one another or other extracellular proteins producing a wide range of effects including the aggregation of PS+ vesicles or membranes (AnxA1, A2, A4, A6 & A7), inhibition of pro-inflammatory cytokine production (AnxA1), regulation of EGF receptor localization/signaling activity (AnxA1, A2, A6 & A8), and even malignant cell transformation, tumor cell proliferation and migration (AnxA2, A3, A4 & A7). Annexin V, a predominately cytoplasmic protein, however, has recently been shown to localize and rapidly (<30 seconds) form a protective protein patch (or plug) at sites of focal cell surface tears. The formation of annexin V patches or plugs prevent the propagation of membrane tears which would otherwise occur due to the high intrinsic lateral surface tension of the underlying peripheral cytoskeleton.

Exogenous annexin V on the other hand, forms a monolayer on regions of externalized PS in the presence of physiologic extracellular concentrations of ionized calcium. These monolayers rapidly assemble into trimers on the cell surface followed by organization into 2D ordered arrays, even at low surface density. As annexin V binds to PS it increases the rigidity of the hydrophobic interior of the lipid bilayer up to the C-12 position of all membrane phospholipids. The observed membrane rigidification appears to parallel the “crystallizing” tendency of PS+ vesicle-bound annexin V. The effect of annexin V binding to the cell membrane is remarkable as one bound annexin V molecule indirectly effects the mobility of 26-28 phospholipid molecules in addition to 4 to 8 directly bound PS molecules. Annexin V molecules also physically interact with one another to change their conformation significantly improving their individual abilities to bind PS (i.e. positive Hill effect) even at low levels of protein occupancy. In contrast antibodies typically require a 70 to 75% occupancy rate of available binding sties before displaying positive cooperativity. Annexin V binding counteracts the loss of membrane rigidity during apoptosis thereby preventing the clustering of PS molecules on the outer leaflet of cell membranes preventing the recognition and subsequent immunosuppressive clearance of apoptotic cells or apoptotic bodies. The buildup of dying tumor cells and apoptotic debris in response to exogenous annexin V has been shown to enhance tumor associated antigen (TAA) recognition as part of the innate and adaptive anti-tumor immune response in multiple models. Annexin V binding may also reduce PS induced enhancement of DGK activity by reversing PS mediated changes in plasma membrane fluidity thereby inhibiting DAG dependent T-cell activation as well as DC maturation. Annexin V's ability to mask (or coat) the surface of PS+ viable tumor cells and exosomes also helps prevent the immunosuppressive actions of PS and PS-opsin receptors expressed by CTLs, DCs and macrophages. The unique features of annexin V listed above make it an attractive agent for cancer immunotherapy and immunodeficiency syndromes including HIV/AIDS and CAR-T cell adoptive treatments.

Annexin V Can Reverse CTL Anergy/Exhaustion of Peripheral Blood Lymphocytes from HIV Patients. A major problem in HIV/AIDS is the inability to clear all reservoirs of virus due to CTL exhaustion/anergy. As HAART (highly active antiretroviral therapy) treatment can only inhibit viral spread and proliferation in dividing cells it can't by itself clear potential reservoirs of virus in quiescent cells such as monocytes/macrophages. Therefore, few HIV/AIDS patients even with stable asymptomatic disease ever clear their chronic infection.

In experiments with peripheral blood sample in patients with HAART treated stable HIV infection, it was found that incubation of exhausted CTL cells in culture conditions with 5 μg/ml of annexin V (1) enhanced HIV-specific CTL expansion (2) that the annexin V induced expansion of HIV-specific CTL was greater than that seen with combined anti-PD-L1 and anti-CTLA4 immune checkpoint therapy (3) no synergistic effect between annexin V and PD-L1 and anti-CTLA4 immune checkpoint therapy (4) annexin V increased granzyme B (a cytolytic protein secreted by activated cytotoxic lymphocytes) in HIV-specific CTL and (5) that annexin Vs effect on CTL expansion is specific to HIV-specific CTL and has no effect on CMV-specific CTL (i.e. non-exhausted) expansion.

For experiments described below, HIV-specific CTL were stimulated with HIV GAG peptide SLYNTVATL. The designations: A03, A07 and A11 are HIV patients. The concentrations used are: 1 μg/ml HIV GAG, 10 μg/ml anti-PDL1 antibody and 10 μg/ml anti-CTLA4 antibody. Granzyme B is quantitated by flow cytometry combined with intracellular staining using fluorescently-labeled anti-Granzyme B antibody.

Experiments with CMV stimulated with CMV peptide (CMV peptide is CMV pp65 NLVPMVATV). The concentrations are: 1 g/ml CMV pp65 peptide, 10 μg/ml anti-PDL1 antibody and 10 μg/ml anti-CTLA4 antibody. The % CMV-specific are measured by FACS assay after incubation of peripheral blood lymphocytes from an HIV+ patient with fluorescent CMV peptide/HLA-1 tetramer and anti-CD8 antibody. CMV/HLA-1 tetramer binds to CTLs which recognize the CMV viral peptide co-presented with HLA-1 antigen (using the viral antigen-HLA-1 tetramer complex to simulate an infected cell).

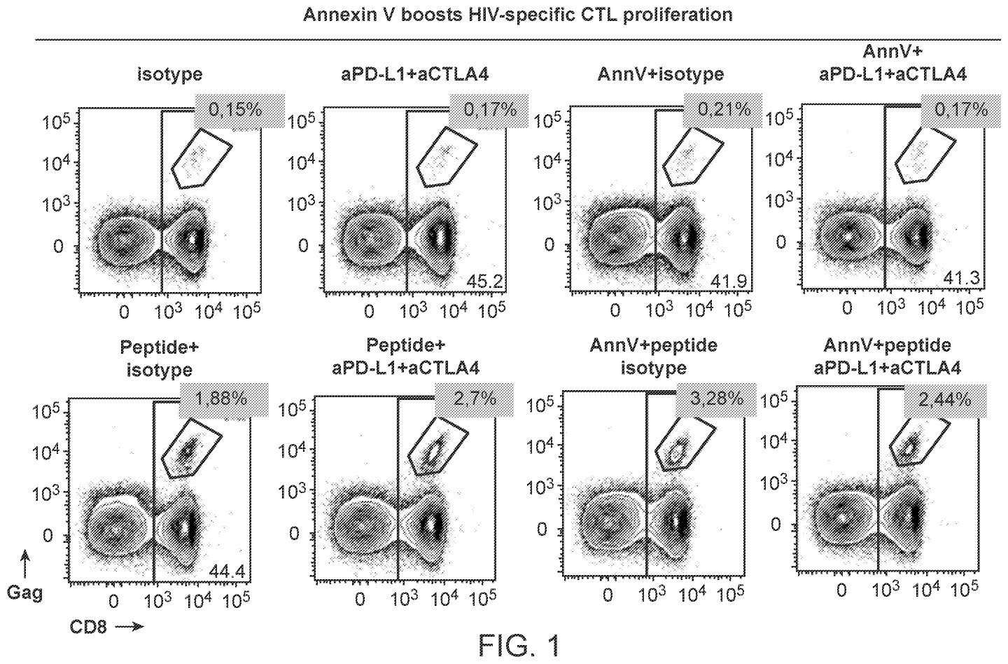

Shown in FIG. 1, Annexin V boosts HIV-specific CTL proliferation. Shown is a FACS assay after incubation of peripheral blood lymphocytes from a patient with HIV with fluorescent GAG/HLA-1 tetramer and anti-CD8 antibody. GAG/HLA-1 tetramer binds to CTLs which recognize the HIV Gag viral protein co-presented with HLA-1 antigen (using the viral antigen-HLA-1 tetramer complex to simulate an infected cell). Anti-CD8 antibody marks CD8+ lymphocytes which are the most common type of cytotoxic T-cell. High double fluorescent staining indicates HIV-specific/CD8+ lymphocytes (marked by boxes, with the percentages of total lymphocyte population in the sample).

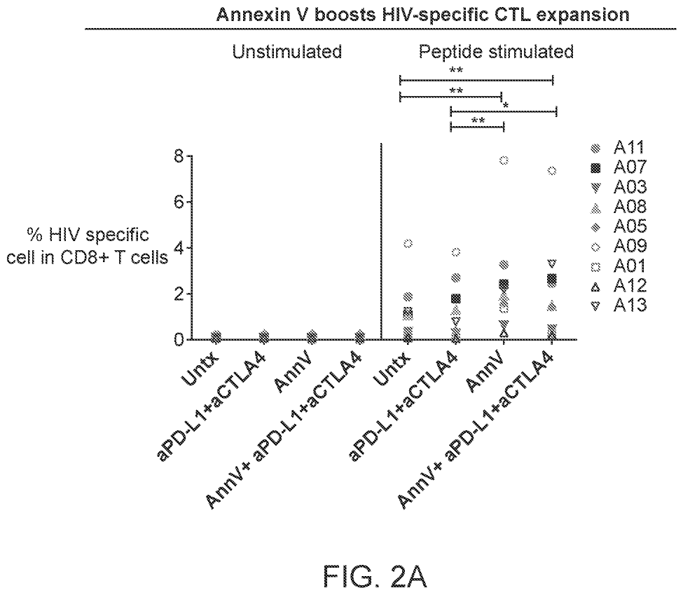

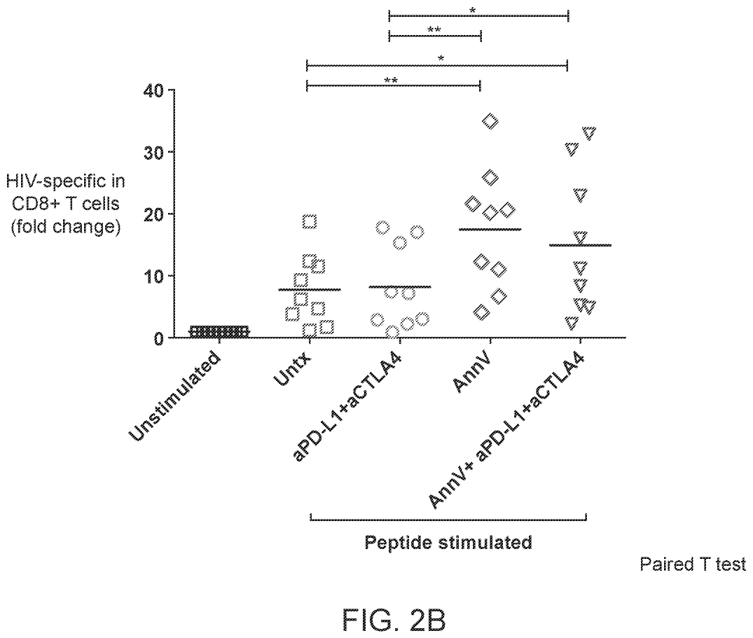

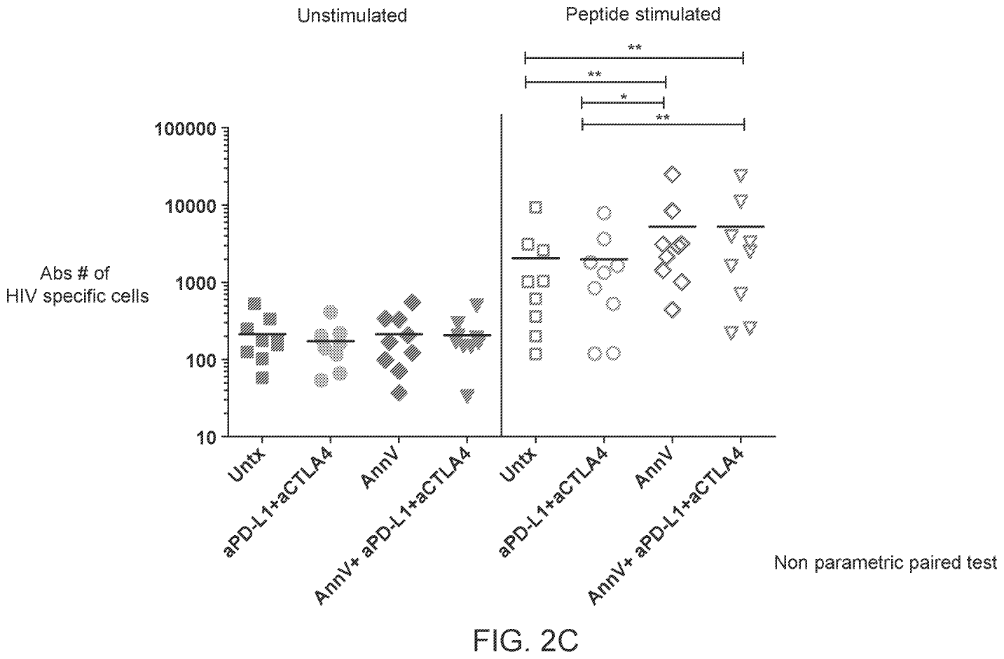

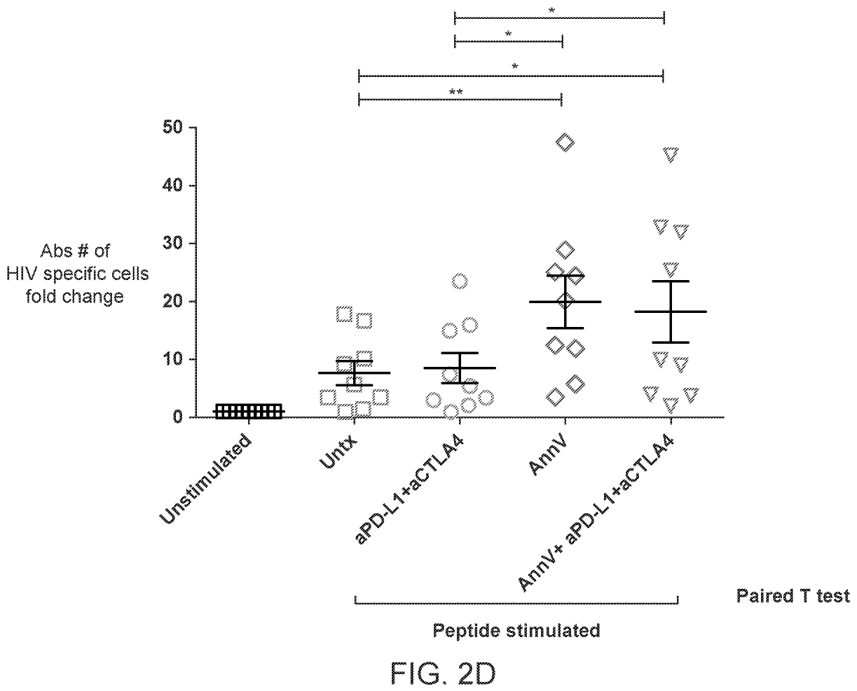

Annexin V boosts HIV-specific CTL expansion, shown in FIGS. 2A-2D. Percentage and absolute numbers of HIV Gag-specific CD8+ T cells after culture for 5 days with HIV Gag peptide with or without treatment with Annexin V or blocking antibodies to CTLA4 or PD-L1. A) The percentages, B) fold increase of percentage, C) absolute numbers and D) fold increase of absolute number of HIV-specific CTLs of nine different HIV patients in response to HIV peptide with and without annexin V, anti-PD-1 & anti-CTLA-4 antibody immune checkpoint inhibition. These data demonstrate that Annexin V increases the expansion of HIV-specific CTL in terms of percentages, increases the expansion of HIV-specific CTL in culture; increases the absolute number of HIV-specific CTL after in vitro stimulation; and increases the absolute number of HIV-specific CTL after in vitro stimulation.

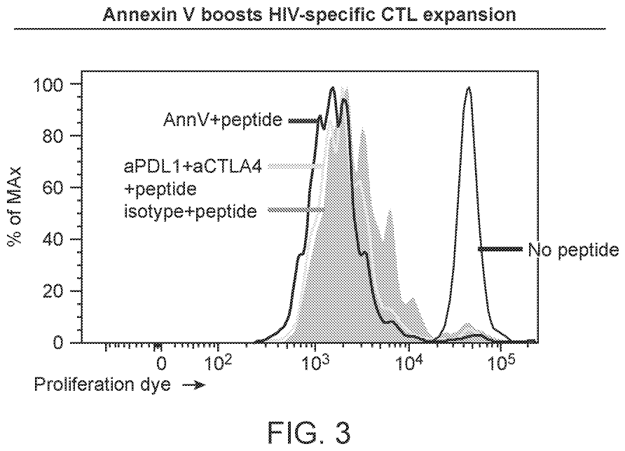

Annexin V boosts HIV-specific CTL proliferation, evidenced by dilution of proliferation dye with decreased fluorescence (dilution of probe, leftward shift of curve) as the HIV-specific CTL population expands in response to HIV-peptide, shown in FIG. 3.

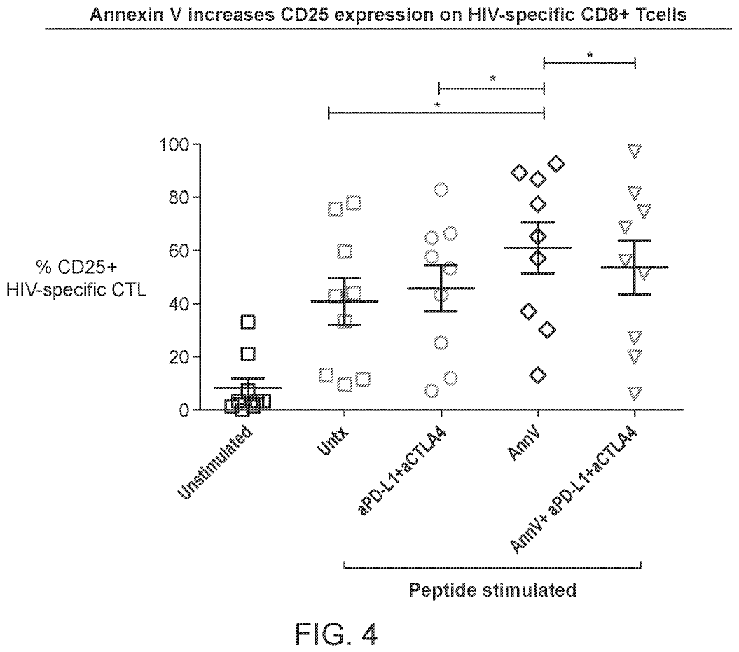

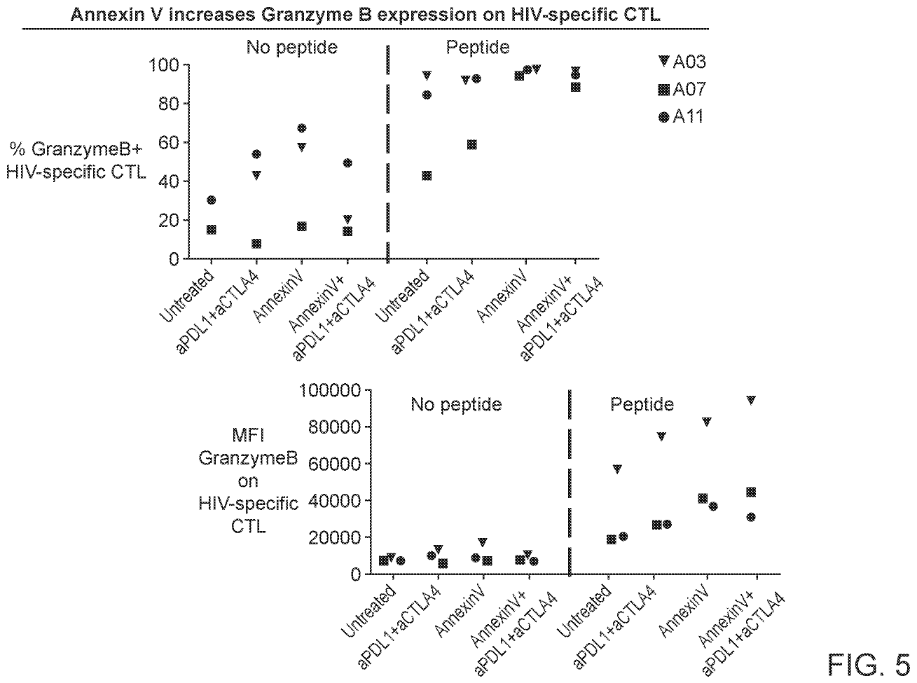

Annexin V increases CD25 expression on HIV-specific CD8+ T cells, shown in FIG. 4. The CD25 antigen represents the α-chain of the IL-2 receptor which is expressed on activated T cells and therefore is a direct marker of T cell activation in this case of HIV-specific CTLs. Data from nine different HIV patients shown comparing the effectiveness of annexin V with HIV peptide stimulation.

Annexin V increases Granzyme B expression on HIV-specific CTL. The activation of HIV-specific CTLs by annexin V results in significant increases in both the number of Granzyme B positive CTLs as well as an increase its total amount, when the cells are incubated with HIV peptide. Granzyme B is a serine protease most commonly found in the granules of cytotoxic lymphocytes (CTLs), natural killer cells (NK cells) and cytotoxic T cells. It is secreted by these cells along with the pore forming protein perforin to mediate apoptosis in target cells.

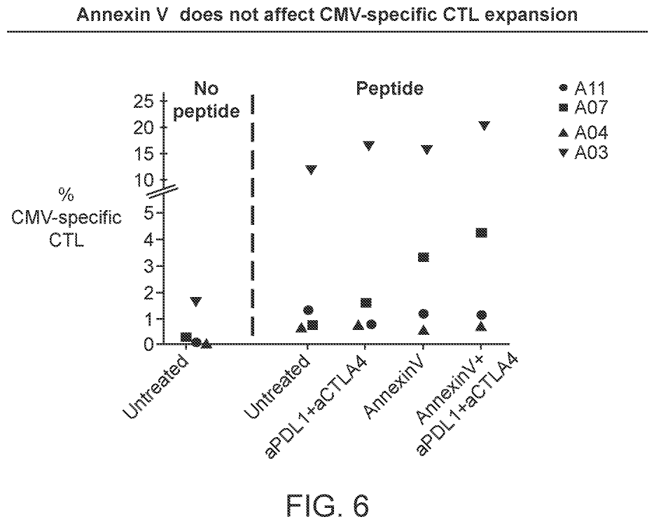

The effect is specific for the HIV exhausted T cells, shown in FIG. 6, where annexin treatment does not affect CMV-specific CTL expansion. CMV-specific CTLs in HIV patients are not exhausted (i.e. anergic) and untreated CMV-specific CTLs proliferate to the same degree as treated CMV-specific CTLs in response to CMV antigen.

The mechanisms may involve multiple PS-dependent mechanisms including the coating of shed PS+ exosomes or apoptotic cells/bodies from viable or dying HIV infected cells with protein, respectively, and the inhibitory effects of increased plasma membrane rigidity after annexin V binding (or internalization) on DGK activity.

1. A method of treating a subject with cytotoxic T lymphocyte (CTL) exhaustion, the method comprising:
administering a course of therapy by parenteral administration of an effective dose of an annexin V agent to reverse CTL exhaustion.

2. The method of claim 1, wherein the annexin V agent is annexin V protein or a phosphatidylserine binding fragment thereof.

3. The method of claim 1, wherein the annexin V protein is human annexin V.

4. The method of claim 1, wherein the subject is a human.

5. The method of claim 1, wherein the CTL exhaustion is associated with a virus infection.

6. The method of claim 5, wherein the virus is selected from Human immunodeficiency virus type 1 and Human immunodeficiency virus type 2.

7. The method of claim 1, further comprising the step of monitoring the subject for clinical signs of infection or for presence of virus following administration of the annexin V agent.

8. The method of claim 1, further comprising the step of monitoring the subject for indicia of CTL exhaustion.

9. The method of claim 1, wherein the effective dose is from about 50 μg/kg to about 20 mg/kg.

10. The method of claim 1, wherein the administering is performed by a route that provides for a circulating half-life of at least about 1 hour.

11. The method of claim 1, wherein the effective dose is administered in a combination therapy with a second agent.

12. The method of claim 1 wherein the subject is also undergoing highly active antiretroviral therapy (HAART).

13. The method of claim 1, wherein the subject has less than 10,000 viral copies per milliliter of blood.

14. The method of claim 1, wherein the effective dose of the annexin V agent results in an expansion CTLs.

15. The method of claim 1, wherein the effective dose of the annexin V agent increases the expression of Granzyme B in CTLs.

16. The method of claim 1, wherein the effective dose of the annexin V agent increases the expression of CD25 in CTLs.

17. The method of claim 1, wherein the CTLs are HIV specific.


SEQUENCE LISTING (3 sequences)

SEQ ID NO: 1 - DNA, 1638 bp, Homo sapiens
agtctaggtgcagctgccggatccttcagcgtctgcatctcggcgtcgccccgcgtaccgtcgcccggctctccgccgctctcccggggtttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaatcagaagcttagccttgaaattgtgaactcttggaaatgttattagtgaagttcgcaactaaactaaacctgtaaaattatgatgattgtattcaaaagattaatgaaaaataaacatttctgtccccctgaattatgtgtacatgtgtgtttagatttattattaaatttatttaacaatgtt

SEQ ID NO: 2 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 3 - protein, 326 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDDAGGCGH